(19) United States
(12) Patent Application Publication
(10) Pub. No.: US 2024/0165415 A1
(43) Pub. Date: May 23, 2024

(54) System Having a Network of Connected Elements Comprising at Least One Medical Device and Method of Operating Same

(71) Applicant: BIOTRONIK SE & Co. KG, Berlin (DE)

(72) Inventors: Rainer Joerg GROSSKOPF, Portland, OR (US); James HORTON, Portland, OR (US); Thorsten WENZLAFF, Berlin (DE); Dirk KLEEBLATT, Berlin (DE)

(73) Assignee: BIOTRONIK SE & Co. KG, Berlin (DE)

(21) Appl. No.: 18/552,573

(22) PCT Filed: Mar. 14, 2022
(86) PCT No.: PCT/EP2022/056424

§ 371 Date: Sep. 26, 2023

Related U.S. Application Data
(60) Provisional application No. 63/167,707, filed on Mar. 30, 2021.

(30) Foreign Application Priority Data
May 17, 2021 (EP) 21173999.0

(51) Int. Cl. A61N 1/372; G06F 9/50; G16H 40/67

(52) U.S. Cl. CPC A61N 1/37282; G06F 9/5061; G16H 40/67

(57) ABSTRACT

The invention is generally directed to a system having a network of connected elements comprising a remote monitoring server (RMS), at least one patient remote device (PR), at least one health care professional (HCP) remote device (CP), and at least one medical device, wherein each of the elements of the system comprises a dedicated communication module, wherein the communication modules of the RMS, the at least one PR and the at least one CP are configured to establish and maintain a communication connection between each other, wherein the communication modules of the at least one medical device and of the at least one PR are configured to establish and maintain a communication connection between each other, wherein each of the system's elements comprises a dedicated processing unit, a dedicated data memory module, and a dedicated energy storage.

### CROSS-REFERENCE TO RELATED APPLICATIONS

This application is the United States National Phase under 35 U.S.C. § 371 of PCT International Patent Application No. PCT/EP2022/056424, filed on Mar. 14, 2022, which claims the benefit of European Patent Application No. 21173999.0, filed on May 17, 2021, and U.S. Provisional Patent Application No. 63/167,707, filed on Mar. 30, 2021, the disclosures of which are hereby incorporated by reference herein in their entireties.

### TECHNICAL FIELD

This invention disclosure describes a system having a network of connected elements comprising a remote server, at least one remote device and at least one medical device.

### BACKGROUND

Medical devices such as implants as known in the art can be coupled with various external devices to exchange data. State-of-the-art implantable pacemakers, cardiac monitors, neuro stimulators and similar implantable medical devices communicate with a health care professional (HCP), e.g., the clinician, using user interface devices (“programmers”) during a face-to-face visit with a clinician. During the face-to-face visit the programmer provides a real-time view of diagnostic and technical data.

In addition, in a wireless monitoring system, a patient device is known to connect wirelessly to a medical device when located near the medical device. Thereby diagnostic and technical data are periodically transmitted to a server. Patient data are downloaded at regular intervals (e.g., daily) from the implantable medical device and forwarded to a service centre comprising the server. At the service centre, the data are pre-processed and, if necessary, transferred to a user, e.g., the HCP.

A patient remote device may, for example, be equipped with a wake-up function to switch an associated implantable medical device from a resting state to an active state in order to establish a communication link with the implantable medical device. The patient device, for example, emits a wake-up signal in a radio frequency (RF) range, which is received by the implantable medical device, whereupon it is activated for data communication with the patient device.

In addition, systems exist providing the possibility for a patient to initiate a recording of data by the implantable medical device. If, for example, a pacemaker patient does not feel well, e.g., if the patient feels dizzy or experiences a tachycardia, the patient can trigger the recording of an ECG by the implantable medical device, e.g., by actuating a remote control. The data recorded in this way is transferred directly to an HCP or to a service centre the next time data is retrieved from the implantable medical device, for example during an examination at the HCP or via an automatic query of the patient remote device. Although some implantable medical devices have means for triggering an interrogation when the patient is remote from an HCP, the patient generally is involved as an actor able to trigger the remote interrogation, the HCP currently having limited ability to trigger the remote interrogation by him-/herself.

It is also known in currently sold products that a patient, who feels symptomatic, may wish to send a report to a clinic, or the clinic may request that the patient generate a report. In either case, the patient follows the process to initiate the patient device to trigger the interrogation. This requires either that the patient places an interrogation wand over the implanted medical device, or if available, the patient device can connect to the implanted medical device via wireless means. The implanted medical device processes the interrogation and transmits data to the patient device. The patient then triggers the patient device to transmit an interrogation message, for example, to a service centre, or if available, the patient device initiates the transmission to the service centre automatically.

In another example, an HCP wishes to have a look at actual patient data and/or data of the patient's medical device in order to prepare for a follow-up of the patient's medical device.

For that, the HCP connects with a service centre and/or retrieves data of the respective patient and the patient's device using his/her remote computer.

However, the data transmission delays in existing remote monitoring designs are too limiting for HCP to provide more complex care to their patients. For example, neuro stimulation systems require interactive feedback from patient to HCP in order to adjust the stimulation therapy effectively. Providing such interactions remotely is not possible with today's systems. In another example, with regard to cardiac rhythm management (CRM), it is desirable to be able to view an intracardiac electrogram (IEGM) and device status in real-time when a patient contacts the clinic and reports the onset of an event. Viewing the IEGM and system data in real-time allows the HCP to accurately triage the patient and determine required next steps. Even though CRM devices are typically capable of detecting and recording events, it should not be assumed that the device has always been correctly programmed to detect the event. As these implanted devices typically transmit data based only upon an internal trigger (e.g., event detection, scheduled update transmission), the data transmitted from them is typically not current at the time of an in-office check. Accordingly, the device is typically interrogated at the start of any office visit, with resulting clinical labor cost and time delay. This function could be performed in advance of the patient visit if interrogation is available to the clinic remotely. I.e., existing solutions require patient and HCP to be in in the same room, in order to view data in real-time and for performing interactive therapy adjustments.

Another state-of-the art system is the Cochlear Limited Nucleus® Cochlear Implant System This system allows for a remote programming session performed via a telemedicine meeting and is intended to reduce the burden to patients who have to travel for appointments. In an in-clinic programming session, a clinical history check and a survey of complaints are performed, as well as tests whose results can pinpoint the needs for new programming. From this information, the programming parameters are chosen, and the precise stimulation levels are determined. To execute this in a remote session using the Nucleus R Cochlear Implant System, equipment must be provided at the patient's location to allow the clinician to both view and hear the patient (i.e., via Skype) so as to monitor their reactions, and a trained HCP must be available at the patient's location to execute the process.

The above state-of-the-art systems have the following drawbacks. Remote data transmission from implanted medical devices to the HCP is delayed, frequently by hours or days after capture. For programming changes, patients must wait until their therapy can be adjusted in a face-to-face session. A medical device has to buffer diagnostic and technical data until it has an opportunity to transmit to an external nearby transceiver. Data storage in the medical device requires electronic memory components that consume significant current and space. For example, cardiac pacemakers and monitors in particular are quite resource constrained, with regard to size and battery longevity. The remote connectivity system connecting a medical device to a remote server is built to a “one size fits all, best case” configuration that in reality may not make the best use of its components for specific use cases. In the field of spinal cord stimulation, the existing solution requires that the patient be present for a face-to-face interrogation by a clinician equipped with a programmer. This requires scheduling and travel by one or both actors (patient and HCP). This allows for a potential disconnect between the onset of symptoms and the implant interrogation. Therefore, the event may be missed. Known devices do not provide for access to real-time data at the time of the event. For CRM, common industry practice requires a patient triggered solution for remote data transmission. This in turn means that HCP must rely on the patient to initiate the data capture and data transmission, and are therefore not in control of how or when the data is captured. Further, as the HCP cannot control receipt of the information transmitted from the medical device, they are not necessarily able to respond to the receipt of the data at the time it is received. This leads to a non-optimal workflow regarding the clinic review of the data. Additionally, the disconnect between transmission of data triggered by the patient and the receipt and review by the clinic can potentially cause a liability situation, if the clinic does not respond in a timely fashion. In the above-mentioned other example for cochlear implants, a “virtual session” must be created for remote data transmission, i.e., all aspects of an in-office interrogation must be recreated in a remote fashion. In particular, the system requires availability of teleconferencing equipment for capturing video and audio feedback during the session. The process also requires a trained person on the patient/remote site that is able to observe and comfort the recipient, confirm communication between the audiologist and the recipient, and to stop stimulation if discomfort is detected. Additionally, it is expected that different combinations and operating modes of equipment, differences in skill level of the remote support person, as well as differences in the telecommunication/internet infrastructure will yield varying results. Having no direct ‘face-to-face’ contact and being dependent on various technologies involved (e.g., video conferencing) will likely confront the audiologist as well as the patient with a highly variable situation.

Accordingly, there is a need to provide a system and method which dynamically and flexibly adapt to different situations and various requirements of the system's users (HCP, patient) and thereby provides optimal performance of the system's elements and modules.

The present disclosure is directed toward overcoming one or more of the above-mentioned problems, though not necessarily limited to embodiments that do.

### SUMMARY

At least the above object is solved by a system having the features of claim 1, a method with the features defined in claim 7, a computer program product with the features of claim 13 and a computer readable data carrier with the features of claim 14.

Particularly, at least the above object is solved by a system having a network of connected elements comprising a remote monitoring server (RMS), at least one patient remote device (PR), at least one health care professional (HCP) remote device (CP), and at least one medical device,
wherein each of the elements of the system comprises a dedicated communication module, wherein the communication modules of the RMS, the at least one PR and the at least one CP are configured to establish and maintain a communication connection between each other,
wherein the communication modules of the at least one medical device and of the at least one PR are configured to establish and maintain a communication connection between each other,
wherein each of the system's elements comprises a dedicated processing unit,
wherein each of the system's elements comprises a dedicated data memory module,
wherein each of the system's elements comprises a dedicated energy storage,
wherein the system provides resources used and shared by the communication modules, processing units, energy storages and/or data memory modules of the system's elements,
wherein the system further comprises a resource manager module configured to determine the resources of communication modules, processing units, data memory modules and/or energy storages involved in a user's and/or the system's at least one requirement, to analyze the determined resources and to automatically control the involved communication modules, involved processing units, involved energy storages and/or involved data memory modules based on the analysis of the determined resources, the current operating modes of the involved communication modules, involved processing units, involved energy storages and/or involved data memory modules and the user's and/or system's at least one requirement to be met.

The system comprises at least one medical device also known as “therapy device”. The medical device may be an implanted medical device (IMD) which may be at least partly implanted in a patient's body. Alternatively, the medical device may be attached to the patient's body or a wearable device. The medical device may provide a therapy to the patient and/or determines bodily parameters from the patient in order to assess their health status and condition. The medical device may be, for example, a pacemaker, defibrillator or another CRM-device, an SCS device or a cochlear implant. The medical device may comprise at least one detector detecting at least one bodily parameter of the patient. The detector is connected to a processing unit of the medical device for processing the determined bodily parameter.

As indicated above the inventive system further comprises the following elements:
A Remote Monitoring Server (or RMS), for example a “Neuro Service Center” (NSC) in one embodiment. The RMS is, for example, a hardware and/or cloud-based service centre that facilitates Remote Programming (RP) and data interrogation of the at least one medical device, and may comprise a central data repository of data collected by the at least one CP, the at least one PR and/or the at least one medical device, wherein the hardware may comprise at least one computing device or a network of a plurality computing devices, wherein the RMS further comprises the computing functionality described herein: the RMS is a functional unit that performs substantial computations, including numerous arithmetic operations and logic operations without human intervention and may comprise, such as, for example, a desktop computer, a server computer, clusters/warehouse scale computer or embedded system: and
At least one Patient Remote (computing) device (PR) may serve as a transceiver to route communication between the medical device and the RMS, and provide to the patient some aspect of local control of the therapy process. For example, it gives the patient the ability to change the active therapy program of a medical device, for example a medical device for SCS, control its stimulation amplitude, turn stimulation on/off, and view battery status. It may also serve as an external programmer for the medical device;
At least one HCP remote (computing) device/programmer (CP) used by HCPs or field technical representatives to communicate with the RMS in order to receive data from the RMS, or to communicate with the PR, in order to, for example, receive data from the PR or to program the medical device remotely. The communication to the PR may be provided via the RMS. The CP may be physical (i.e., dedicated computing device) or virtual (e.g., via web-based interface to the system, secure connected data reporters).

For communication, public or private communication network(s) (PCN) is (are) used and in each CP an HCP device user interface (CUI), e.g., GUI at the physical CP or web portal at virtual CP and for each medical device a PR user interface (UI). The RMS may further manage deployed at least one PR and at least one CP, which are together called external devices (ED) in the following.

In one embodiment the PR remote device and/or the HCP remote device may be a smartphone or tablet comprising an app realizing above and below described functionality.

For data/signal processing, each of the CP, RMS, PR, and medical device comprises a dedicated processing unit, which is generally regarded as a functional unit of the respective element, that interprets and executes instructions comprising an instruction control unit and an arithmetic and logic unit. The processing unit may comprise a microprocessor, a controller, a digital signal processor (DSP), an application specific integrated circuit (ASIC), a field-programmable gate array (FPGA), discrete logic circuitry or any combination thereof. Alternatively or additionally, the processing unit may be realized using integrated dedicated hardware logic circuits, in particular in the case of a medical device due to the small size and extreme power limitation. The processing unit may comprise a plurality of processing subunits.

Each element of the system comprises a dedicated communication module for uni- or bidirectional communication as indicated above. The communication is mainly extracorporeally, with the medical device it may be partly intracorporeally, if the medical device is an IMD. The communication is provided by one communication technique wirelessly via the air using electromagnetic waves, for example, EDGE, EV-DO, Flash-OFDM, GPRS, HSPA, LoRaWAN, RTT, UMTS, Narrowband IoT, Bluetooth, WLAN (WiFi), ZigBee, NFC, LTE, Wireless USB, Wibree (BLE), Ethernet or WiMAX in the radio frequency region, or IrDA or free-space optical communication (FSO) in the infrared or optical frequency region. Accordingly, the respective communication protocols, often with the same name (i.e., a system of rules that allows two or more entities of a communications system to transmit information via an kind of variation of a physical quantity, defining rules, syntax, semantics and synchronization of communication and possible error recovery methods and being implemented by hardware, software or a combination of both) or cooperating protocols (protocol suites) may be used, for example TCP/IP protocol suite (e.g., IPv6, TCP, UDP, SMTP, HTTP/2) also with TLS or SSL, IPX/SPX, X.25, AX.25, ebXML, Apple Talk, Bluetooth protocols (e.g., BR/EDR), Bluetooth 4.0 (BLE), ZigBee protocol, NFC protocol, IEEE 802.11 and 802.16 protocols, etc. The communication module comprises a sender, a receiver and/or a transceiver for sending and/or receiving the respective communication signals. The communication module is electrically connected to the processing unit of the respective element or may be integrated within the processing unit. The communication module may be electrically connected to a data memory unit of the respective element as well. The communication module may provide at least two communication paths, for example three or more communication paths, wherein each communication path uses a different one of the above-mentioned communication techniques based on one specific communication protocol. As two different communication paths are regarded:
communication techniques/communication protocols which work basically different such as, for example, Zigbee and Bluetooth, and
different versions of the same communication technique/communication protocol such as, for example, Bluetooth 4.0, Bluetooth 4.1, Bluetooth 4.2, and Bluetooth 5, IEEE.11b, IEEE.11g, IEEE.11ax.

Alternatively, two different communication paths are provided by two communication paths which are based on the same or compatible communication protocol but have a different service set identifier (SSD). The service set is a group of wireless network devices which share an SSD—typically the natural language label that users see as a network name. A service set forms a logical network of nodes operating with shared link-layer networking parameters, the form on logical network segment.

In particular, each medical device comprises a communication module in order to send data determined by the medical device to and/or to receive data from the at least one PR. In one embodiment, the communication module of the medical device has a sender or a transceiver that uses a pre-defined communication path using a pre-defined communication protocol in order to uni- or bidirectionally communicate with the PR. Accordingly, the communication module of the PR is configured to communicate with the medical device analogously. In contrast, the communication modules of the RMS, the at least one PR and the at least one CP are configured to establish and maintain an above described communication connection between each other.

Accordingly, the communication module of each element of the system comprises the respective hardware and software adapted to the communication techniques/communication protocols which are provided by the respective communication module. Further, it is necessary that the two or three of the elements RMS, PR and CP comprise communication modules providing at least partly the same communication path (RMS and at least one remote device) in order to “talk” to each other, i.e., to establish and maintain a communication connection between each other in order to transmit data, for example data of a bodily parameter of the patient and/or data of the medical device implanted within or attached to the patient's body. Sometimes the communication techniques are downward compatible which means that a communication module with a higher version of a communication protocol “understands” a communication module with lower versions of a communication protocol, i.e., a working communication connection can be established by communication modules comprising different versions of the same communication module. Such situations shall be covered by the present invention.

Further, the communication module of each element comprises a measuring unit or detector for different connectivity parameters such as, for example, at least one parameter of the group containing network speed, ping time, packet loss percentage, transmission latency, transfer/transmission rate, signal strength, success rate of communication for each of the implemented communication paths of the respective communication module. The communication module determines continuously or in pre-defined time intervals values of the respective connectivity parameter(s).

The PR remote device and/or the HCP remote device may ping the respective other device via the RMS or the RMS during the maintenance of a communication connection at regular intervals to ensure that handshakes persist and that any delays are minimized.

In one embodiment, the communication module is configured to store the determined values of the at least one connectivity parameter together with the location and/or time of determination by the respective PR, CP and/or RMS, for example, a GNSS value of this location. Accordingly, the determined values of the at least one connectivity parameter are linked to the respective location information and/or time information. This allows to favor one communication path based on the actual location and/or time for the at least one remote device at least as a first approximation.

Further, each element of the system comprised the data memory module which may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The data memory of the RMS is referred to as central data repository in the following, as well.

Each element of the system further comprises a dedicated energy storage for power supply, e.g., a battery.

The communication module, data memory module and energy storage of each element of the system are electrically connected to the processing unit and thereby with each other, wherein the communication module, the processing unit and the data memory module are configured to communicate data/signals with each other.

Further the system provides resources used and shared by the communication modules, processing units, energy storages and/or data memory modules of the system's elements. These resources may be, for example, the above-mentioned connectivity parameters of the present or to be established communication connections or (different) communication paths, and parameters derived therefrom, the charge level of the individual units, resolution of transmitted data (e.g., sampling rate, sampling granularity), used data memory capacity.

There may also be minimum level settings that each component can't drop below, in the case where it will be asked to share excess capacity. These may occur in the implant, and consist of minimum resources that must be reserved for therapy operations: e.g., battery level, memory capacity, processing capacity.

Additionally, the system further comprises a resource manager module configured to determine the resources of communication modules, processing units, data memory modules and/or energy storages involved in a user's and/or the system's at least one requirement. This requirement may be a use case of the system, for example, an interrogation of the medical device required by the HCP using their CP, an automatic, system-driven (e.g., regular/continuous) interrogation of the medical device requested by the PR, remote or personal programming of the medical device, patient-triggered report sent from the PR to the CP of the HCP (e.g., if the patient feels symptomatic), the RMS periodically collects technical data of the medical device of the patient and values of bodily parameters of the patient determined by the medical device, etc. Each (automatic) system's requirement and each user's requirement (HCP, patient, maintenance technicians) involves at least part of the elements of the system—which depends from the chosen specific requirement. In the first step, the resource manager determines the actual values of the resource or the plurality of resources, for example the resource(s) which are relevant to the specific requirement.

Further, the resource manager knows the respective, actual operation modes of the involved communication modules, involved processing units, involved energy storages and/or involved data memory modules, e.g., the actual communication path used by the communication connection between CP and RMS. The resource manager module further analyzes the determined resources and automatically controls the involved units (i.e., the involved communication modules, involved processing units, involved energy storages and/or involved data memory modules) based on the analysis of the determined resources, the current operating modes of the involved units and the user's and/or system's at least one requirement to be met.

In one embodiment, the inputs are used for the control algorithm(s) (for example, a “Random Forest”) comprising at least one parameter of the group containing:
Component specifications of the involved resources,
Minimum allowable resources availability (e.g., minimum battery SoC requirement for device therapy operation, minimum memory available, etc.),
Current resources utilization,
Use case in operation,
Networks available,
Current measured network speed(s),
Historical measured network speed(s),
Current ping response time,
Current packet loss percentage,
Current data transfer rate,
Current signal strength,
Global Navigation Satellite System (GNSS) location,
Time at current location,
Historical success rate of communication for each of the available communication paths of the respective communication module. This will be used as a weighting factor in choosing the communication path, i.e., communication paths with a high average level of success will be weighted higher than those with a lower average success rate.
Selection as to whether the communication module shall determine continuously or in pre-defined time intervals values of the respective connectivity parameter(s). This is the means by which the system determines whether it needs to change connectivity routing.

In the embodiment where only a single device is making the selection of connectivity path and resource allocation, its weighting is “1”. In embodiments where multiple devices are involved in the decision process, each individual device has a weighting of <1 in the decision process, where the sum of all weightings equals 1. Individual weighting is based upon the relative “value” of that component in the overall system, as determined during system design and testing.

The decision making systems (AI/Algorithms) may provide for the following purposes:
To automatically and dynamically select and maintain optimum connectivity path for a remote monitoring system fulfilling a specific use case. Patient input requirement should be zero and shall be minimal.
The system is able to choose the best connectivity path and resource allocation at a specific time based on current measurement of the values listed above, with historical performance factored into the selection.
The system continuously monitors actual performance and can change routing based on current actual performance.
Upon successful completion of the use case, the system automatically and dynamically reverts system resources back to the default configuration, or adapts to a new demand based upon use cases in queue.

This means that the system reacts dynamically (instead of statically in the state-of-the-art systems), reacting to the user's intentions and/or needs of the current operating environment of the system. This can be accomplished by approaching resources as a system-wide shared resource, with individual device behavior changing based upon the current use case, i.e., the current system and/or user requirements. For example, the resource manager module may solve—mathematically speaking—an optimization task or similar mathematical problem which may be derived from the present user and/or system requirement and the available resources of all involved units and controls all involved system's elements based on the result of this optimization task or similar mathematical problem with regard to the determined resources of the involved units of the whole system.

A successful system typically requires a balancing act between multiple competing (and potentially conflicting) system demands. In particular, the medical device as one part of the system is resource constrained (due to focus on minimal size, optimum therapy features and maximized time between battery changes or charges), and frequently has to be configured as a static tradeoff between data transmission latency and data volume. The medical device is designed to be configurable to a hoped-for “best case” trade-off of several quality aspects, e.g., longest period of connectivity loss without permanent loss of data, size of electronic data storage in the medical device, battery longevity (i.e., data transfer protocol is optimized to meet described use cases with minimal battery impact), data resolution (sampling rate, sampling granularity). Another part of the system, e.g., the communication connection(s) to other system elements require different behavior adapted to the specific system's and/or user's requirement. For example, some use cases require that no data in the stream is lost, while timely delivery is not required (e.g., pace/sense counters, arrhythmia episodes, IEGM snapshots for an arrhythmia episode). For this data stream the medical device's data memory module has to be large enough to buffer data until the medical device gets a chance to deliver it to the next hop in the pipeline, such as a PR. In other use cases it is more important that the data sampled is delivered to the user with minimal delay, but it is less important that the dataset is complete (e.g., when transmitting real-time streaming IEGM samples, or current heart rate). For this data stream the medical device's data memory module only needs to be large enough to buffer the data for the maximum allowed delay. Older data can reasonably be purged, if the medical device did not previously get a chance to deliver it to the next hop in the pipeline.

The resource manager module is a module comprising hardware and/or software that is configured to provide the functionality described herein. The (main) resource manager module may be located centrally, for example, within the RMS but may alternatively or additionally comprise local submodules or part modules within at least part of the elements of the system. In one embodiment, the resource manager module comprises a part module within each element of the system. The resource manager (part) module may be integrated within the processing unit of the respective element of the system or comprise an own processing unit that is connected with the (main) processing unit of the respective element. If there are part modules and submodules they are connected, also, if applicable, to the main module of the resource manager module and form a network in which determined resources are exchanged and analyzed (evaluated) together over all involved elements with regard to a specific system or user requirement.

It is important that by approaching resources as a system-wide shared resource the individual behavior of the elements of the system are changed based upon the system's and/or user's requirements. For example, data memory module's and processing unit's usage may be pushed to a portion of the system where less restrictions on available resources exist (e.g., to the RMS instead of the medical device). In another example, data memory module's and processing unit's usage may change or may be reapportioned within a particular system element depending upon the system needs of the current use case. In another example, the transmission rate at a particular system element may be altered based upon the current demands of the communication and/or the battery “budget” for a data transmission of the medical device may be adjusted based upon the priority of the information.

In one embodiment, the resource of the communication modules of the system's elements comprise at least one of the above-mentioned connectivity parameters of at least one communication path through which the communication connections of the respective communication modules are provided. The resource manager module may receive the values of the connectivity parameters from the communication modules of the system's elements. Further, automatic control of the communication modules comprises, for example automatic change of the communication path and/or bandwidth, if the currently used communication path between two elements of the system and/or the bandwidth is not appropriate to the system's and/or user's requirements according to the analysis of the resource manager module.

In one embodiment, the resource of the processing units of the system's elements is the load of the respective processing unit and/or at least one critical parameter. The critical parameter is described below with regard to an implanted loop recorder but may be analogously used for other systems. The critical parameter is a parameter that is determined as or from at least one bodily parameter of the patient which exceeds or goes below a pre-defined threshold value and thereby triggers a pre-defined requirement to the system.

In one embodiment, the resource of the data memory modules of the system's elements comprise the storage space of the respective data memory module.

In one embodiment, the resource of the energy storages of the system's elements comprises the charge level of the respective energy storage.

In one embodiment the resources of similar units (e.g., energy storages of different, involved elements) or different units (e.g., energy storages and communication modules of all involved elements) of the system may be combined in the above explained analysis of the resources in order to fully assess the system's behavior with regard to the user's and/or system's requirements.

In one embodiment for use in connection with a CRM device as a medical device a pacemaker may stream the current heartrate over 24 hours, 7 days a week at low resolution (i.e., averaged per min), with long latency and “guaranteed” delivery, delivering this data to the next hop, the PD, once per day. This makes sense when the user's intention is to remotely monitor the patient over many months, with minimum impact to battery life. However, if the same patient sees the HCP face-to-face, the pacemaker would switch to streaming the current heart rate in a high resolution/low latency mode, but allows for dropping data that is older than 1 minute, and for a greater draw on the battery. This makes sense when the HCP wants to monitor the current state of the patient, and the patient's heart rate from a few minutes ago is not relevant, wherein the storage location switches based upon patient's intention/use case requirements. Both modes use the same implementation in the medical device, but the system's operation is automatically tuned for different resolution and different frequency of data delivery to the next hop in the pipeline, based upon the needs of the current situation.

In this CRM embodiment, the next hop in this data streaming pipeline is PR-to-RMS. Here the system may also adapt to the user's intention. For remote long-term patient monitoring, the PR batches the heartrate data stream into daily chunks before delivery to the RMS. In addition, during a face-to-face patient/HCP interaction, the PR adapts to forwarding the heartrate data stream to the next hop (RMS) as quickly as possible, and the RMS forwards it again as quickly as possible to the CUI of the CP. Again, both modes use the same implementation in the PR and the RMS, but it is automatically tuned for different frequency of data delivery to the next hop in the pipeline.

In one embodiment, for use in a system having an SCS device as medical device, in addition to long-term remote monitoring and face-to-face acute monitoring, the system may accommodate a third use case, wherein the HCP interacts in real-time with a remote patient. This embodiment would allow the HCP to assess the patient's current status, review the current device programming, and transmit suggested programming changes to the patient, all during the period of a single phone call. This mode requires both low latency and high data resolution, even though the patient is not face-to-face with the clinician.

In one embodiment, for use in an Implanted Loop Recorder (ILR) or similar CRM medical device, the system may allow for a Systemic Inflammatory Response Syndrome (SIRS) alert with high urgency. Based on values of bodily parameters determined by at least one sensor provided by the medical device (e.g., temperature, respiratory demand, heart rate, activity level, white blood count) the processing unit of the medical device recognizes, for example, that a value of critical parameter determined by the medical device exceeds or goes below a pre-defined threshold value of a critical parameter (e.g., a critical parameter for SIRS). The critical parameter may be a bodily parameter or a parameter calculated or derived from at least one measured bodily parameter. For example, the processing unit thereby determines that a systemic infection is likely occurring. Accordingly, the communication module of the medical device and of the PR, RMS and CP are controlled such that an alert signal is produced by the communication module of the medical device and that this alert signals is transmitted via PR, RMS and CP to the HCP with high urgency/priority over other signals. The example may operate as follows. The detector of the medical device may track temperature, respiratory demand, heart rate and activity level over time, and establishes a baseline for the specific patient. If a relevant shift in these baseline measurements occurs that is consistent with a systemic infection (i.e., increase in temperature, respiratory demand and heart rate, but with a decrease in activity level) the resource manager module requires that the medical device captures the relevant information and transmits the data immediately, as an alert signal with highest urgency/priority via the PR, RMS and CP to the monitoring clinic. The resource manager module may additionally force the medical device to provide a closer monitoring of the parameters named above by increasing the frequency of measurements and to send them to the RMS e.g., for storing the values and/or further analysis. Such method may be programmed in the medical device at the time of an in-clinic follow-up, or configured remotely via a command sent from the RMS. Further, the resource manager module may force the RMS to provide for emergency alert channels to the CP designed to quickly bring clinical attention to the alert (e.g., special SMS/text, email and automated voice message channels). Accordingly, the monitoring clinic would then take steps to further triage the information to confirm the root cause of the issue. This embodiment may be used for infections as common as an infected implant site, or as extraordinary as a COVID response.

In another CRM embodiment, similar to the example above, an existing CRM medical device with Closed Loop Stimulation (CLS) and an accelerometer may be configured to detect an increased respiratory demand at rest, which would then trigger the resource manager module to produce an alert signal and other suitable measures. For example, the resource manager module or the medical device establishes a baseline for the patient using the CLS and accelerometer data. If the system detects a sustained demand per the CLS algorithm, but no corresponding increase or even a decrease in accelerometer activity, the resource manager module forces the medical device to capture the relevant information and transmit the data immediately, as an alert with highest urgency, to the monitoring clinic. In one embodiment the resource manager module may be able to respond to emotional demand; i.e., the increased demand and reduced activity levels must persist for a longer period of time in order to trigger the alert.

The resource manager module—though not as sensitive a trigger as what can be provided with a medical device with a higher number of sensors—may be used as dynamic means that initially triages for infections as common as an infected site of the medical device, or as extraordinary as a COVID.

In one embodiment, the control of the resource manager module may provide a feedback loop in order to increase the effectivity and dynamics of the control.

In each of the above scenarios, the system is able to automatically and dynamically alter its behavior to match the current system needs. No operator input is required to switch or adjust the system. In one embodiment, each unit or element in the system is able to recognize the system's and/or user's requirement (use case) being requested of it, communicate the new requirement to the other elements of the system's network as needed. The resource management module optimizes all necessary elements for that scenario.

In contrast, in this invention, the elements of this system and their units dynamically and automatically interact in order to provide the optimal environment for the requirements of the user and/or the system, for example, facilitate a continuous data stream between elements. The system may utilize a networked “decision tree” within the communication infrastructure that communicates to the individual elements as to what the current requirement is, and how to behave during the respective use case. This optimizes each element in the chain of connected subsystems capability for the specific requirement, e.g., maintaining a continuous connection to its neighbors, for example, the medical device and PR maintain a Bluetooth connection while they are in proximity, PR and RMS maintain a TCP/IP network connection over the Internet while mobile networks are available, CP and RMS maintain a TCP/IP network connection over the Internet while mobile networks are available and the HCP is in proximity to the CP with its CUI. In one embodiment, new data are transmitted to the next hop along the chain as soon as the new data is available. This reduces the latency of the transmission to (nearly) zero, such that no time delay is perceived by the HCP. Therefore, data from the medical device may be displayed on the CUI in real-time. This allows, for example, showing a stream of ECG or IEGM data in real-time on the CUI while they are captured on the medical device.

In one embodiment, the control of resource management module controls the storage space of the data memory modules of the system's elements such that a data buffer of sufficient depth is provided to sustain occasional loss of connectivity. This allows to send new buffered data as soon as connectivity to the neighbor is restored. Further, the resource management module frees up local data storage as soon as the neighbor has received the buffered data.

In a CRM embodiment, for example, at the beginning of a CRM face-to-face follow-up session the CP sends a command to the medical device which changes the parameters of the heart rate stream to low latency, high resolution, and reduced buffer depth. At the end of the face-to-face follow-up the CP sends a command to the medical device which changes the parameters of the heart rate stream back to high latency, low resolution, and “guaranteed” delivery. Additionally the medical device does the same an hour or so after losing connection to the CP.

In one embodiment, the system may be used for triggering a real-time interrogation of the patient's medical device current data, and for remote analysis of current and historical patient data, all done during a communication session between the CP and the medical device via the RMS and the PR.

The present invention may be used in multiple areas of medical device application, e.g., spinal cord stimulation (SCS), cardiac rhythm management (CRM), and many other medical fields that utilize an implanted medical device e.g., deep brain stimulation (DBS), occipital nerve stimulation (ONS), trigeminal nerve stimulation (TNS), vagal nerve stimulation (VNS), phrenic nerve stimulation, gastric electrical stimulation, and sacral nerve stimulation (SNS).

The following features are also/may also be implemented in any combination with each other:
HCP-triggered request for the interrogation of the chosen medical device, wherein the interrogation may be initiated remotely from one or more CUI, e.g., (1+N) CUI where N is a natural number: N=0, 1, 2, . . . .
The data collected by the interrogation may be viewed remotely at (1+N) UI or CUI.
Data derived from interrogation are a combination of subjective and objective data.
Data derived from interrogation are a combination of current and historical data.

In one embodiment, one or more (e.g., all) steps in the sequence of establishing communication connections may require a “handshake” process that may: 1.) ensure cybersecurity, and 2.) provide automatic data persistence, communication persistence and communication repetition to ensure that the communication between each element is completed.

The medical device's, in particular, the implantable medical device's, communication module, data memory module, energy storage, and processing unit may be contained within a hermetically sealed housing. This ensures particularly reliable protection of the medical devices against external interference (e.g., moisture).

For remote programming (RP) or interrogation using the CP, in one embodiment the use of single, serial handshakes between each element of the system is significantly reduced. Each element is designed to autonomously maintain a continuous connection to its respective neighbours, via an on-going session that persists for the duration of the RP or interrogation process, wherein the medical device has bidirectional communication link to the PR, the PR has a bidirectional communication link to the RMS and the RMS a bidirectional communication link to the CP, for example:
Medical device and PR maintain, for example a Bluetooth connection while they are in proximity.
PR and RMS maintain, for example a TCP/IP network connection over the internet while mobile networks are available.
CP and RMS maintain, for example a TCP/IP network connection over the internet while mobile networks are available.

Further, feedback loops may exist between the elements to maintain a closed loop of one element with the element adjacent to it in the communication pathway, to proactively assess the status of the communication process and to automatically and immediately process information when received. Each link between one element to the next transmits new data to the next hop in chain as soon as new data is available, thereby reducing transmission latency to (nearly) zero. Additionally, in case of connectivity loss, any system element may send new buffered data, if necessary, via a newly established communication path as soon as connectivity to the neighbour is restored. The system, i.e., each of the at least one CP, the RMS and at least one PR comprises a data buffer of sufficient depth to sustain occasional loss of connectivity, and to allow the transmission of large datasets.

The RMS may be hosted by a service provider, e.g., a company external from a clinic. The RMS may comprise at least one of the following services:
Repository of data collected by ED and medical devices;
Repository of keys and certificates for communication of the elements of the system and the HCP or patient via UI or CUI with the system;
Facilitates remote programming by routing data to/from their intended destinations;
Communication with an external push notification service(s)
CP fleet management (e.g., via Airwatch and processes to manage CPs);
Medical device instance authentication service;
HCP, technician and patient authentication service; and
Database analytic tools.

As indicated above, the RMS may serve as a repository of data collected by Eds and medical devices stored in its data memory module (also called central data repository). The medical devices may comprise home monitoring (HM), which originates from the medical device and is routed to the RMS via the PR. In one embodiment the HM data may be encrypted by the medical device and may only be decrypted by the RMS. In this embodiment, the PR does not have any key to decrypt the data. RMS's “certificate and key management system” (CKMS) may be used to decrypt the data.

Further, after the above mentioned communication connections are established from the CP to the chosen medical device as indicated above, the RMS is used as the conduit to route RP commands from the CP to the medical device and/or return responses and status updates from the medical device to the CP.

A hardware and/or cloud server (e.g., CouchbaseR server) may be used to store the data in the RMS. The RMS notifies Eds to pull data from the server via push notifications. RMS sends a push notification request to an external notification service (i.e., Microsoft Azure, Google Firebase Cloud Messaging), which in turn is sent to a specific ED.

In one embodiment, a subset of data is copied from the central data repository of the RMS to a data warehouse to support internal business functions such as technical forensics, quality monitoring, and clinical studies.

CKMS may be used to generate and store secrets, e.g., encryption keys and authentication credentials, for every manufactured medical device. Secrets are generated during manufacturing and stored into the medical device. Generated secrets are used to cipher data when:
Remote programming data is sent from the RMS to the medical device;
HM data are sent from the medical device to the RMS.
RMS uses an authentication management and access service, e.g., Keycloak, for PR authentication. During provisioning, a PR creates a username and password on the authentication management and access service. Subsequently, PRs submit their credentials to the service for an access token, for example, based on JSON Web Token (JWT). The token is then presented to RMS Sync Gateway to gain access. This follows the OAuth 2.0 authorization code grant model. OAuth is an open standard for access delegation, for example, used as a way for Internet users to grant websites or applications access to their information on other websites but without giving them the passwords.

The RMS may provide CP user management and authentication using a Microsoft Azure Active Directory (AAD) which may be integrated in the RMS or may be realized as an external unit connected with the RMS. In the latter case, the RMS interacts with AAD for HCP and patient management operations.

In one embodiment, CPs may follow the OAuth 2.0 authorization code grant model to gain access to the RMS, where AAD acts as the authorization and token endpoints. Once authenticated, the CP is given an access token, which in turn passes to RMS Sync Gateway to gain access.

Medical devices may send/receive data to/from the RMS, via the PR, such that the PR may not decipher the data. Each medical device has unique keys assigned to it at the time of manufacturing.

Eds may communicate to the outside world via above mentioned public communication connections. This introduces a large attack surface that may impact the ED-to-medical device programmability.

However, CPs may be protected by the following:
Malware protection and firewall (e.g., Windows Defender malware and firewall).
CP users are granted user rights and thus not able to install software.
Internet resources are whitelisted to resources that are needed for its functionality (e.g., NSC, Microsoft, MDM provider, etc.)
Access to other internet resources, such as Microsoft Store, is inhibited or limited to privately published apps.

PRs may be protected by the following:
No internet browsers installed.
Messaging and phone apps disabled.
Access to other internet resources, such as Google Play Store, is inhibited or limited to privately published apps.

In one embodiment, CouchbaseR may be used as the database engine used to store and share data in the RMS. Eds may use Couchbase Mobile, and the RMS may use CouchbaseR Sync Gateway and CouchbaseR Server. This setup allows for data replication between the mobile client (Eds) and the RMS.

On the PR, information may be stored e.g., in the CouchbaseR instance, pertaining to the medical device, which is associated with such aspects as therapy program settings, battery state of charge (SoC), connection state, etc. This data, in turn, is replicated at the RMS.

In one embodiment data transmission between ED and RMS is encrypted, as well as communication link between the ED and medical device. For example, a 2-factor authentication is used when the CP user (e.g., the HCP) selects a chosen patient's medical device to remotely program. In one embodiment, the RMS is configured to store and/or manage the authentication and/or encryption keys and/or certificates for the at least one CP, at least one PR, at least one medical device as well as for the at least one user of the system, for example, patient, HCP and/or technician. For that, the RMS may integrally comprise hard- and/or software solutions for that or the RMS may be connected to respective hard- and/or software solutions. For example, Microsoft Azure may be responsible for managing this on the backend, and Microsoft APIs may be responsible for managing user input on the CP side (i.e., login mechanism, communication with Azure, handling of 2-factor input, etc.).

In one embodiment, once the CP user has been authenticated, an authentication service of the system returns one unique signed token, e.g., the above mentioned JWT token, to the CP (referred to as the remote programming session token). The CP provides this token to the RMS during every remote programming transaction during the remote programming session. The one unique token is valid for the duration of the current remote programming session: subsequent remote programming sessions require a new unique token, which means the HCP having to go through the authentication sequence again. Using such tokens allows decentralizing authentication and authorization.

In one embodiment, the at least one PR may be not configured to continuously poll the RMS for changes, as that would result in unnecessary overhead on both the PR and RMS (i.e., data usage from the PR's perspective and request processing from the RMS's perspective). Instead, the RMS may interface with an external push notification system, such as Google's Firebase Cloud Messaging system, to send a notification to the target PR corresponding to the chosen medical device. Conceptually, for example, prior or at the beginning of establishing a communication connection or at the beginning of the remote programming session the target PR receives a push notification from the RMS. This notification serves as a trigger to the PR to poll the RMS server at a faster rate. In one embodiment, the notification does not contain detailed information, such as any identifying or secret data.

In one embodiment, a remote programming session begins with the CP user (e.g., the HCP) logging into their CP. Regardless of a session type, all CP users must log in. After logging in, the CP user will be able to select the option to start a remote programming session, which will present them with a list of patients they may remotely program.

From a hierarchal standpoint, patients may be organized by their clinic. CP users may be associated with one or more clinics. Therefore, CP users may only remotely program patients' medical devices for clinics that they are associated with.

The following data may be transmitted during a communication session (i.e., “signals”). Please note that these data will differ dependent upon the embodiment. The following data may refer partly to a communication within an SCS system which is described here as a typical embodiment.

a) Medical Device Data
Medical device data (serial number, medical device type)
Therapy data (e.g., therapeutic data (packed parameter values, packed data elements, and interface parameters), lead impedance, offset measurements, therapy program selection, stimulation on/off, enable/disable MRI mode, battery SoC (it is device data, but it tells you whether or not you can deliver the therapy), status changes, lead info/configuration (“blob” and interface data), medical device alerts, IMD EC App Status, stimulation usage data (used for statistics)
Personal data (patient name, physician name, hospital, implantation date of the medical device, indication, lead position, lead type and extension information, implanted system element traceable IDs
Notes
Patient Identifier (unique ID, serial number of medical device)

b) Technical Data
Technical data/logs (reset, battery, etc.)
System-based events
Battery history log
MRI mode
Magnet history
medical device reset log
ED application log
application logs
Error logs
Audit logs

The RMS as configured transmits at least part of these data from the RP while the RMS transfers data with the CP.

In one embodiment, for remote programming of the chosen medical device, the CP is configured to produce a single program containing the updates and/or changes of the medical device's parameters and to transmit the single program to the medical device via the RMS and the corresponding PR. Further, in one embodiment the RMS is configured to encrypt the single program received from the CP, wherein the corresponding PR is configured to transmit the encrypted single program to the chosen medical device. Accordingly, the PR cannot decrypt the single program. Decryption is provided by the medical device.

In one embodiment, the RMS is configured such that its central data repository comprises data having different security level, wherein one example of data with a lower security level are pseudonymized data. Data with a higher security level are personal or private data such as names, addresses, user ID etc. These data need higher protection and can only be accessed by persons having a higher security level.

Further, at least the above object is solved by an operating method of a system having a network of connected elements comprising a remote monitoring server (RMS), at least one patient remote device (PR), at least one health care professional (HCP) remote device (CP), and at least one medical device,
wherein each of the elements of the system comprises a dedicated communication module,
wherein the communication modules of the RMS, the at least one PR and the at least one CP establish and maintain a communication connection between each other, wherein the communication modules of the at least one medical device and of the at least one PR establish and maintain a communication connection between each other,
wherein each of the system's elements comprises a dedicated processing unit,
wherein each of the system's elements comprises a dedicated data memory module,
wherein each of the system's elements comprises a dedicated energy storage,
wherein resources are used and shared by the communication modules, processing units, energy storages and/or data memory modules of the system's elements,
wherein the system further comprises a resource manager module which determines at least one resource of communication modules, processing units, data memory modules and/or energy storages involved in a user's and/or the system's at least one requirement, analyzes the determined resources and automatically controls the involved communication modules, involved processing units, involved energy storages and/or involved data memory modules based on the analysis of the determined resources, the current operating modes of the involved communication modules, involved processing units, involved energy storages and/or involved data memory modules and the user's and/or system's at least one requirement to be met.

In one embodiment, the resource of the communication modules of the system's elements comprise at least one connectivity parameter of at least one communication path through which the communication connections of the respective communication modules are provided, wherein connectivity parameters are, for example, network speed, ping time, packet loss percentage, transmission latency, transfer/transmission rate, signal strength, success rate of communication, resolution of transmitted data.

In one embodiment, the resource of the processing units of the system's elements comprises the load of the respective processing unit and/or at least one critical parameter.

In one embodiment, the resource of the data memory modules of the system's elements comprises the storage space of the respective data memory module.

In one embodiment, the resource of the energy storages of the system's elements comprises the charge level of the respective energy storage.

In one embodiment, wherein the control of the resource manager module provides a feedback loop.

The above embodiments of the data transmission method have the same advantages as the above system. Embodiments of the system indicated above may be realized in the data transmission method analogously. It is referred to the above explanation of the system in this regard.

The above method is, for example, realized as a computer program which comprises instructions which, when executed, cause the processing units (processors) of the elements of the system to perform the steps of the above method which is a combination of above and below specified computer instructions and data definitions that enable computer hardware to perform computational or control functions or which is a syntactic unit that conforms to the rules of a particular programming language and that is composed of declarations and statements or instructions needed for a above and below specified function, task, or problem solution.

Furthermore, a computer program product is disclosed comprising instructions which, when executed by the processing unit, cause the processing unit to perform the steps of the above defined method. Accordingly, a computer readable data carrier storing such computer program product is disclosed.

The above system and method are configured to maintain connectivity between subsystems whenever feasible. Further, using the resource manager module the elements of the system are configurable. As indicated above, the system adapts dynamically to user's actions (e.g., HCP or patient) or environmental demands. Further, the system may provide an availability of an “interrogation on demand” that is continuous for the duration of the session and occurs in real-time. The session may be triggered by the HCP or by the patient. Generally, the system increases availability of data access, improves clinic workflows, and reduces travel demands on patients to achieve desired care. For the use as a CRM system it has the advantage that no patient interaction is required as long as the PR is close enough to the implanted medical device and has connectivity and that control of data capture is improved.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawings, wherein:

FIG. 1 shows one embodiment of the inventive system and the steps establishing the communication between the elements of the system;

FIG. 2 depicts another sketch of the inventive system;

FIG. 3 shows a remote programming overall workflow;

FIG. 4 shows the flow of events for transmission of data to the PR during RP;

FIG. 5 depicts an example of 2-factor authentication using push notification based approval;

FIG. 6 depicts a flow diagram of initiating an RP session (CP to PR);

FIG. 7 shows a sequence diagram of initiating an RP session (CP to PR);

FIG. 8 shows a flow diagram of RP session, namely PR to CP response;

FIG. 9 depicts the RP session communication CP to PR to medical device;

FIG. 10 shows a sequence diagram of RP with transmission acceptance:

FIG. 11 shows a sequence diagram of RP with transmission rejection: and

FIG. 12 shows a sequence diagram of RP transmission with connection loss.

### DETAILED DESCRIPTION

In the following, embodiments of the inventive system will be explained in detail. The embodiments are explained with regard to medical devices in the form of spinal cord stimulation (SCS) devices and a remote monitoring server (RMS) in the form of a Neuro Service Center (NSC) and with regard to remote programming which may include data interrogation.

FIG. 1 shows the elements of the system, namely the RMS 1, which is, for example, the NSC, at least one HCP remote device (CP) 3, at least one patient remote device (PR) 5, and at least one medical device, for example an SCS device, 7. For further information and for the properties with regard to the system elements 1, 3, 5, 7 it is referred to above explanation in the general description.

The RMS 1 comprises a communication module 22a, the CP 3 a communication module 16a, the PR 5 a communication module 17a and the medical device 7 a communication module 18a. Each communication module 16a, 17a, 18a, 22a is configured to provide a communication connection using different communication paths, for example to communication connections via mobile wireless networks (e.g., GPRS data connection, UMTS data connection, LTE data connection), virtual private network(s) or dedicated line(s), TCP/IP via IPv4 and IPV6 (or WLAN), internet and local networks via Ethernet, electronic patient/case files (electronic medical records) via HL 7 v2 or v3 or similar. Further, suitable communication standards e.g., TLS+TCP/IP, SSL+TCP/IP or ebXML may be used. The communication module of the respective element of the system is electrically connected with its own (main) processing unit and data memory, wherein the data memory module of RMS 1 is denoted as central data repository 21. Further properties of each communication module 16a, 17a, 18a, 22a is explained in the general part of the description above.

An overview of a remote programming (RP) method in which communication connections provided by the communication modules 16a, 17a, 18a, 22a are used may be derived from FIG. 1. The remote programming method comprises the following steps. At first, the HCP triggers the communication session at his/her UI (CUI) at the CP 3 by a session request 11 which is sent to the RMS 1. Then, the RMS initiates a bidirectional communication connection 12 with the PR 5 based on a handshake with the PR 5. In the next step, the PR 5 initiates a communication connection 13 with the medical device 7 based on a handshake process, as well. Then, the medical device 7 sends a response to the CP 3 via the PR 5 to RMS 1 to CP 3 communication chain which is denoted in FIG. 1 with reference number 14. Now a continuous bidirectional communication connection is established between the CP 3 and the medical device 7 with all elements now connected in real-time. Upon completion of the use case, the session initiator (HCP) indicates at CP 3 that the session is complete and signals the other elements to release their handshakes (step 15). Throughout this process, only one handshake is required between adjacent elements, each communication channel stays open and steady until a closing message is sent which closes all connections between the elements.

The RMS 1 further comprises a processing unit 22c and an energy storage 22d, which provides power supply if the connection to the power network which normally provides the power supply of the RMS 1 is lost. The CP 3 comprises a data memory module 16b, a processing unit 16c and an energy storage 16d, for example a battery. The PR 5 comprises a data memory module 17b, a processing unit 17c and an energy storage 17d, for example a battery. The medical device 7 comprises a data memory module 18b, a processing unit 18c and an energy storage 18d, for example a battery. The units/modules of each of the system's elements 1, 3, 5, 7 are electrically connected, for example the communication module 16a, the data memory module 16b, the processing unit 16c and the energy storage 16d. The units/modules of the elements of the system may have the properties explained in the general part of the description above.

Further, each processing unit 16c, 17c, 18c, 22c comprises a resource manager module part 16e, 17e, 18e, 22e, wherein all parts of all elements 1, 3, 5, 7 of the system together form the resource manager module. The resource manager module parts 16e, 17e, 18e, 22e communicate with each other and exchange resource data determined in each element of the system 1, 3, 5, 7, respectively. The resource manager 16e, 17e, 18e, 22e determines at least one resource of communication modules 16a, 17a, 18a, 22a, processing units 16c, 17c, 18c, 22c, data memory modules 16b, 17b, 18b, 21 and/or energy storages 16d, 17d, 18d, 22d involved in a user's and/or the system's at least one requirement (use case), analyses the determined resources and automatically controls the involved communication modules, involved processing units, involved energy storages and/or involved data memory modules based on the analysis of the determined resources, the current operating modes of the involved communication modules, involved processing units, involved energy storages and/or involved data memory modules and the user's and/or system's at least one requirement to be met.

In one embodiment during establishment of the communication connection the respective communication module 16a, 17a, 18a, 22a determines values of communication parameters (see examples above), transmits them to its processing unit 16c, 17c, 18c, 22c. There, the communication parameters are analysed by the respective resource manager module part 16e, 17e, 18e, 22e because the communication parameters form resources of the system as indicated above. With regard to the requirement of the HCP, that a remote programming of the medical device 7 shall be realized, the respective resource manager module part 16e, 17e, 18e, 22e (after sharing the data with the other resource manager module parts) arrives at the result to use the following communication protocols/communication paths:
Medical device 7 to PR 5: Bluetooth Low Energy (BLE)
PR 5 to public communication network(s) (PCN), e.g., 4G LTE, WiFi 802.11
PR 5 to RMS 1: TCP/IP
RMS 1 to public network(s): VPN
CP 3 to RMS 1: WLAN

Further, the resource manager module part 16e of CP 3 determines that if, for example there are different WLAN networks available in the area of CP 3, the communication module 16a at CP 3 uses the strongest WLAN-connection. Further, if, during the maintenance of the communication session the actually-used WLAN connection drops out, the communication module 16a automatically switches to the next-strongest WLAN-connection available. The resource manager module 16e, 17e, 18e, 22e continuously determines the resources of the system's elements (e.g., connectivity parameters, actual battery status of energy storages 16d, 17d, 18d, 22d) and analyses these data in order to decide whether the control of the resource manager module 16e, 17e, 18e, 22e needs to change/update operation of the respective element of the system.

For example, if the resource manager module 16e, 17e, 18e, 22e of the system detects that battery charge of the energy storage (e.g., battery) 18d of the medical device 7 has dropped below a pre-defined limit for the medical device 7 or that the battery charge of the storage (e.g., battery) 17d of the PR 5 has dropped below a pre-defined limit value for the PR 5, it will provide a low charge notification to the patient via the PR 5 UI. Further, the resource manager module 16e, 17e, 18e, 22e reduces transmission frequency/payload from the affected element, until the low charge is resolved.

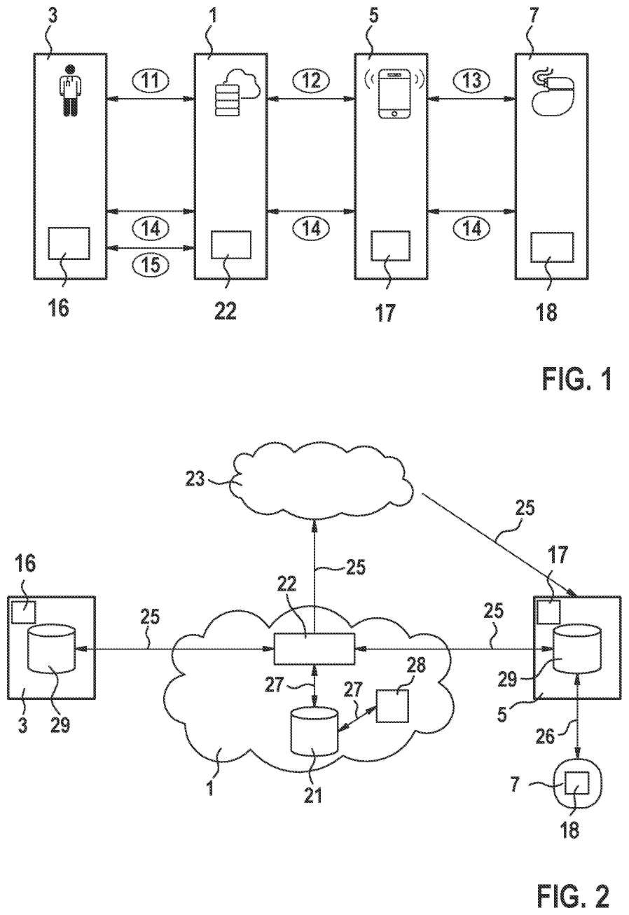

FIG. 2 contains a different visualization of the whole inventive system. The RMS 1 comprises a Couchbase server 21 as central data repository to store data. Additionally, RMS 1 comprises a Couchbase Sync Gateway as the communication module 22a for synchronization and communication with CP 3 and PR 5. The system further comprises External Push Notification Services 23 (e.g., Microsoft Azure, Google Firebase Cloud Messaging) in order to notify the CP 3 or the PR 5 to pull data from the RMS 1 via push notifications. For that, the RMS 1 sends a push notification request to the external push notification services 23. In FIG. 2 the arrows 25 represent communication connections as indicated above, the arrow 26 a BLE connection and the arrows 27 local network connections (e.g., LAN connections). The RMS 1 further comprises a certificate and key management system (CKMS) 28 which is used to decrypt the data received from the at least one medical device 7. In one embodiment the External Push Notification Services 23 may be integrated in the RMS 1. Further, in order to communicate with the Couchbase server 21 of the RMS 1 the CP 3 and the PR 5 each comprises a Couchbase mobile application 29.

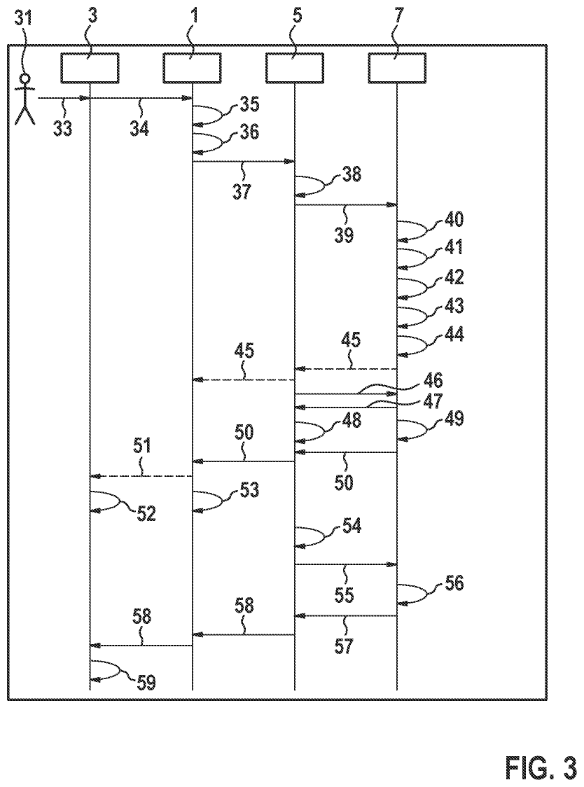

FIG. 3 shows an overall workflow for remote programming which is explained in further detail in the following. The workflow for remote programming works provided the continuous communication connection is previously established between the CP 3 and the medical device 7 after selection of a specific patient/medical device 7 for remote programming. During the continuous communication connection, it may be possible, that the network drops out or that the connectivity parameters determined by the involved communication modules move into a region in which a continuous connection is not guaranteed according to actual or historical data. Then, the respective resource manager module part 16e, 17e, 18e, 22e automatically switches in a well-ordered manner to another communication path providing a more reliable connection, if available, so that continuous communication is provided. “Continuous communication” with regard to the embodiment presented means that the communication connection is upheld but the communication path used may be switched. Alternatively, if the resource manager module part 17e of the PR 5 or the resource manager module part 18e of the medical device 7 detects that the connectivity level has dropped below a preset limit for the PR 5 or medical device 7, respectively, the resource manager module part 17e of PR 5 will provide a low connectivity notification to the patient via its UI, and initiates a “handshake” protocol that reduces transmission frequency/payload, until the low connectivity is resolved. As indicated above, the resource manager module part 17e of PR 5 also has the ability to scan between available connectivity options to select the best option for the current scenario.

The sequence of events starts with the CP user (e.g., an HCP) who commands the transmission of a therapy program using remote programming. As indicated below, the therapy program contains all changes/updates of the parameter of the medical device 7 which are to be programmed. The method describes the process uplink and downlink.

At first, after authentication of the HCP 31 at his/her CP 3 the HCP 31 configures a new therapy program for the patient to be used at its medical device 7. Then, after pressing the transmit button (step 33) the therapy program is transmitted to the RMS 1 (step 34). At the RMS the access token is verified (step 35) and a remote package containing the information of the therapy program is ciphered (step 36). This remote package is then transmitted (automatically, i.e., without further HCP or patient interference) to the PR 5 (step 37). In the next step, the patient is prompted about the fact that there is a therapy program arrived at his/her PR 5 (step 38). In the next step 39 the patient accepts the therapy program and the ciphered remote package is automatically sent to the medical device 7. The remote package is not deciphered at the PR 5 but at the medical device in step 40. In step 41 the therapy program is saved to a buffer of the medical device 7. Step 42 comprises ruling a check to the therapy program in medical device 7 and step 43 and installation and activation of the therapy program. Additionally, in step of 44 the medical device 7 generates and ciphers a remote acknowledgment signal which is to be sent to the RMS 1.

Then, using the continuous communication connection to the RMS 1, the encrypted acknowledgment signal is returned to the RMS 1 via the PR 5 (see steps 45).

Additionally, the PR 5 may automatically interrogate the medical device 7 for new data. This is facilitated in step 46, wherein this step contains an interrogation request to the medical device 7. This interrogation request may be sent via the continuous communication connection between the PR 5 and the medical device 7. In response to this request in step 47 the medical device 7 provides the PR 5 with its interrogation data. Then, in step 48 the PR 5 updates its graphical UI. At the same time the medical device 7 generates and ciphers HM PGMR frame in step 49. The PGMR frame contains detailed parameter values programmed in the medical device. In step 50 the PGMR frame is returned to the PR 5 and routed to the RMS 1. Here, the authenticity of the programmed parameter values can be checked. In the next step 51 the latest therapy entities may be pulled to the CP 3. Then, in step 52 the CUI of the CP 3 is updated and at the same time in step 53 the PGMR frame deciphered, data assembled, PPVs unpacked and PDEs as well as appropriate entities at the RMS 1 updated. This allows to check the success of the remote programming procedure on the CP and to give feedback to the HCP via the CUI.

In the next step 54 further adjustment of the medical device 7 may be provided by the PR 5, for example, in the case of an SCS a global amplitude may be adjusted. A respective signal, for example, to adjust the medical device's program stimulation amplitudes and to save the global amplitude may be sent to the medical device 7 in step 55. The respective action is provided by the medical device in step 56. In step 57 a response is sent from the medical device 7 to the PR 5 returning stimulation amplitudes. The following steps 58 returns the latest status of the PR 5 to the RMS 1 and the CP 3. Finally, in step 59 the CUI the of the CP 3 is updated accordingly.

In the process described with regards to FIG. 3 the steps 34, 37, 51 and 58 may be Couchbase, commands and responses. Further, the steps 39, 45, 50 may be provided as end-to-end encrypted commands and responses.

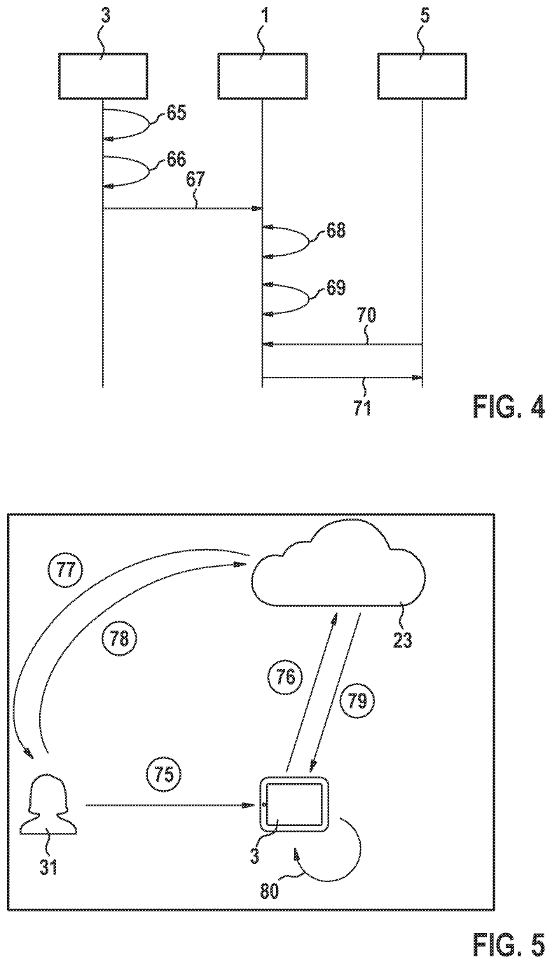

FIG. 4 describes the first steps of remote programming using the Couchbase server 21 and Couchbase mobile 29 of the CP 3 in more detail. When CP 3 commands a change to a target medical device 7 (step 65), the changes/updates of the parameters of the medical device 7 are packaged in step 66. Then, they are placed into the local a Couchbase instance by the Couchbase mobile 29 and pushed to the RMS Couchbase sync Gateway 22a and the Couchbase server 21 (step 67). Then, the package or its information is encrypted using the shared keys of the target medical device 7 provided by CKMS 28 (step 68). Because the medical device's shared secret key is only known by CKMS 28 and the medical device 7, the PR 5 is not able to decipher the package's contents. Then, the encrypted remote package is placed into the Couchbase application (step 69). The Couchbase changes are pulled and sent at the next polling interval after it has finished synchronizing with the RMS's Couchbase server 21 (see steps 70 and 71 in FIG. 4).

Prior to above programming steps the remote programming process needs to be initiated which is explained in the following with regard to FIG. 5 showing the start of a remote programming (RP) session after the HCP was logging into his/her CP 3.

Patients may be organized by their clinic. HCP, i.e., CP users, may be associated with one or more clinics. Accordingly, it may be defined that a CP user is allowed to remotely program patients medical devices only for such clinics to which they are associated. In one embodiment, a 2-factor authentication is required to be provided if the HCP selects a patient for remote programming. As indicated above, Microsoft Azure may be responsible for managing this on the back end and Microsoft APIs may be responsible for managing user input on the CP side (login mechanism, communication with Azure, handling of second factor input etc.).

The RP session starts with the HCP requesting to start remote programming in step 75 in FIG. 5. Then, in step 76 user credentials are passed for a remote programming token to the external push notification service 23. In step 77 this service sends an authorization request to the HCP's mobile device and in step 78 the HCP 31 accepts the request. Then the external push notification service 23 returns a signed access token to the CP 3 in step 79. The process then continues with remote programming (step 80).

The token (MFA token) provided by the external push notification service 23 to the CP 3 is a signed JWT token which is also referred to as the RP session token or access token. The CP 3 provides this token to the RMS 1 in every RP transaction during the RP session. The token is valid for the duration of the current RP session, subsequent RP sessions require a new token which means having to go through the authentication sequence described above again.

In the following steps, the elements of the system execute connectivity steps. For that, the following messages/information are utilized by the system and/or the method:
Follow-up ID (a unique identifier assigned to each session, for process function and tracking)
The above-mentioned multifactor authentication token (MFA, i.e., the token of the RP session determined by 2-factor authentication).
RpStartSession message: an entity requesting the start of a remote programming sequence and contains information to specify the target patient. This is used when the HCP selects a patient on the CUI for the remote session. RMS's remote-programming service triggers the associated PR 5 with a push notification as explained above. The PR 5 may start a replication, in case there are a lot of un-replicated data to catch up with.
RpCpRequest message: containing the therapy program parameters
RpPrRequest message: containing encrypted programming commands
RpRmsStatus updates CP 3 with programming progress: captures the status of RMS's processing of the RP request with various timestamps, etc. This message contains, for example, time of RpCpRequest received, time of RpCpRequest signed by CKMS, time of RpPrRequest generated, time of push notification to PR sent, other state and error info
RpCpStatus. The RpRmsStatus and RpPrStatus entities are used to communicate status at each hop. Each entity may contain tasks that each hop is responsible for performing, errors/status values, completion of tasks denoted by a timestamp.

The remote programming method further makes use of a “Time to Live” calculation (TTL) in its control process. As indicated above, TTL is a value in an Internet Protocol (IP) packet that tells a network router whether or not the packet has been in the network too long and should be discarded. This TTL is an absolute time in UTC. TTL is based on PR's 5 time to account for time synch mismatches between CP 3 and PR 5. TTL is calculated using current PR time acquired at session start and elapsed time since RMS data base synchronization and timeout interval. When PR receives RpPrRequest entity, it takes the TTL value and starts a timer. Expiration of TTL is communicated by setting the respective value in the RpPrStatus entity.

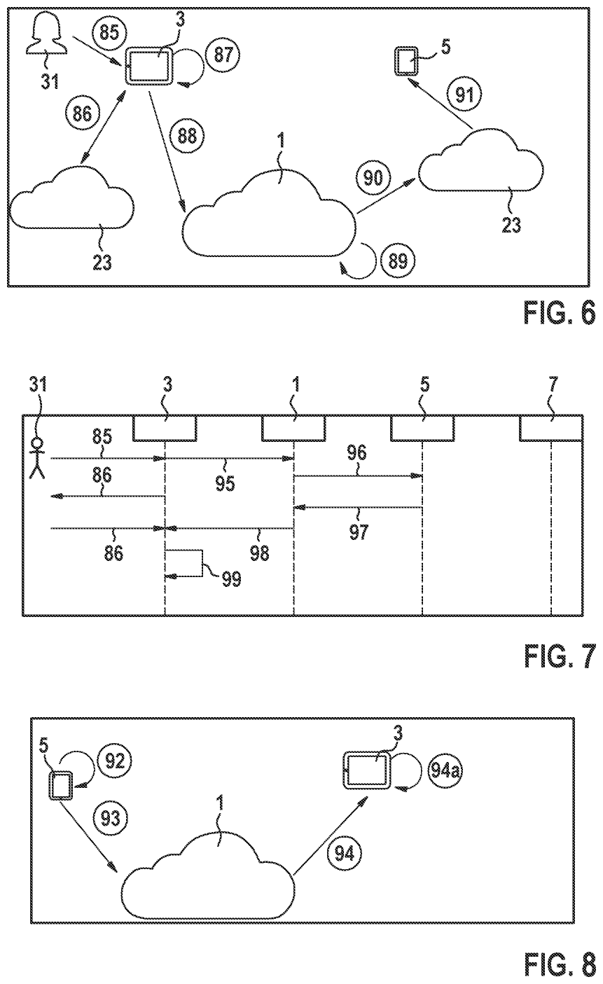

With regard to FIG. 6 further steps during initiation of an RP session are explained. This flow diagram shows that the PRs 5 are not configured to continuously poll the RMS 1 for changes as that would result in unnecessary overhead on both the PR 5 and the RMS 1. Instead, the RMS interfaces with the external push notification service 23 to send a notification to the target PR 5. For example, at the beginning of a remote programming session the target PR 5 receives a push notification. This notification serves as a trigger to the PR 5 to poll the RMS 1 (i.e., its Couchbase server 21) at a faster rate. The notification does not contain detailed information such as any identifying or secret data.

For example, the following steps may be executed for remote programming (i.e., remote follow-up). At first, the HCP selects a patient to program in step 85. The HCP navigates to the remote patient management page on the CUI found on the CP 3 and selects a patient from a filterable list of opted-in patients who the HCP is authorized to follow-up with. Then, the 2-factor authentication is provided as indicated with regard to FIG. 5 (step 86). At the same time, the CP 3 creates an entity requesting the start of an RP session (step 87), i.e., the CP 3 sends a RpStartSession command to the RMS. This means that an uplink is created from the CP 3 to PR 5. The CP 3 then waits for successful authentication, and for the PR 5 to respond with an object of the medical device 7 of the patient via RMS 1. Then, the RpStartSession is synchronized with the RMS 1 (step 88). After that, the RMS 1 identifies the target PR 5 which corresponds to the selected patient (step 89), e.g., using Google firebase. Then, a push notification request is sent to the external push notification service 23 (step 90), which contacts the PR 5 corresponding to the selected patient and the PR 5 receives the push notification and is thereby triggered to replicate with the RMS 1 at a faster rate (step 91).

As a response, which is depicted in FIG. 8, the PR 5 synchronizes with the RMS 1 (step 93) after the current PR time and medical device connection state was placed into a response entity (step 92). For example, the current battery state, therapy data such as stimulation state, and/or MRI mode state, and any medical device errors or other data of the PR database will be sent from the PR 5 to the RMS 1. This information is also used for the continuous determination of resources provided by the resource manager module 16e, 17e, 18e and 22e. Similarly, the CP 3 synchronizes with the RMS 1 (step 94), wherein the above-mentioned data are further transmitted to the CP 3. Additionally, the CP 3 quotes data from relevant entities to start the remote programming session (step 94 a). Updates to this information will be delivered on a change in any of these items. If the CP user (e.g., HCP) has not begun editing a program, the PR will also update the CP via RMS 1 with any changes to the active program, program strength, or program start strength. Relevant status information will be logged using the normal logging mechanisms. If the response of the PR 5 is received within a pre-defined time (e.g., 30 seconds) of the CP 3 sending the RpStartSession command, and the response indicates that PR is connected to the medical device and has the expected follow-up ID, the CP enters the standard programming page in remote programming mode. Otherwise, CP displays an appropriate message and stays in the remote patient management page.

FIG. 7 shows the start of the remote programming session as a sequence diagram which is explained in the following. As indicated above the HCP 31 first selects a patient who needs to receive a program update of his/her medical device 7 (step 85). The 2-factor identification illustrated above is provided by the steps 86. Then, a start session command sent from the CP 3 to the RMS 1 (step 95). In the next step 96 the RMS 1 sends a start session notification to the PR 5. Triggered by this command the PR 5 responds by sending information received from the medical device 7 previously (step 97). This information, for example, may comprise current battery state, stimulation state, MRI mode state, or any medical device errors. This information is further transmitted to the CP 3 in step 98. Further, CUI is updated in step 99 afterwards.

In the remote programming mode CP application stages parameter changes provided by the HCP 31 to a single therapy program using the programming CUI. Remote programming differs from face-to-face in that instead of transmitting changes immediately pending changes will be highlighted to indicate to the HCP 31 the changes that will be made. Attempting to switch programs prompts the HCP as the CP user to discard his/her changes or stay on the current program. If the HCP has made all desired changes and taps the transmit button a ‘transmission in progress’ overlay may be displayed to prevent navigation or further program edits. This overlay may persist until programming completes will be HCP chooses to end the session.

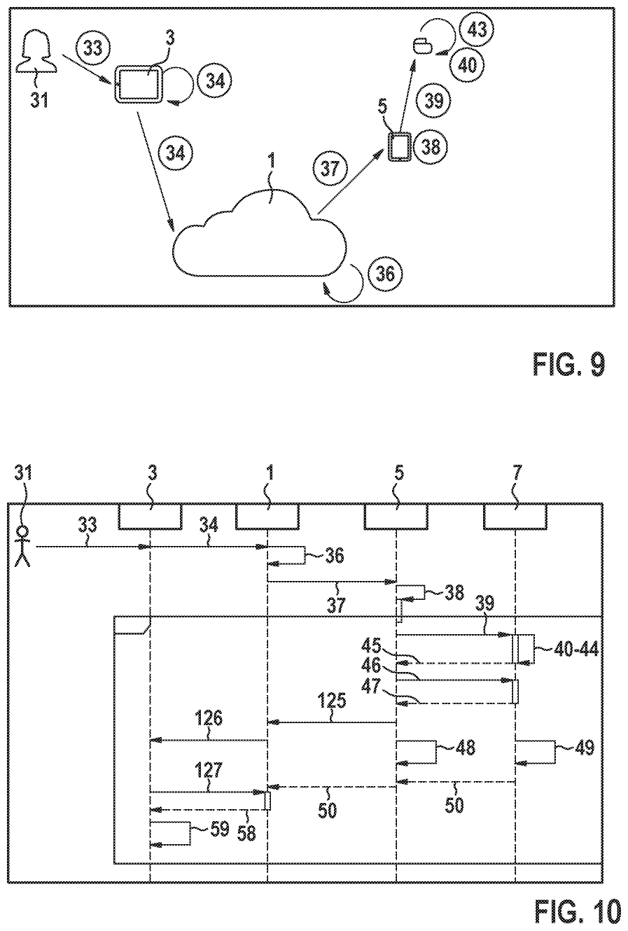

The following steps of remote programming are visualized in FIGS. 9 and 10. Pressing the “Transmit” button starts the remote programming using the established communication chain CP-to-RMS-to-PR-to-medical device. The following steps describe uplink and downlink.

For the safety and comfort of the patient, all programs updated through remote programming will have their program strength set to a pre-defined minimum value. After successful programming, the patient may be required to adjust the medical device parameters, for example the strength and default strength to an appropriate level, using the standard PR 3 UI. If the remote programming session is still in progress, these changes will be reflected in the CP 3, as described above.

After pressing the transmit button (step 33, see FIG. 10), to perform the transmission, the CP 3 creates and sends an RpCpRequest entity, containing the therapy program parameters, to the RMS 1 (step 34). After program encryption (step 36), the RMS 1 then sends an RpPrRequest to the PR 5 containing encrypted programming commands (step 37) and creates an RpRmsStatus to update CP 3 with programming progress. The RMS 1 starts a Push Notification to the PR 5 so that the PR 5 may start a replication to get the RpPrRequest update as indicated above. The PR 5 does not parse this data. Upon receiving this request, if the TTL has not expired, the PR 5 asks the patient to accept or decline the program update (step 38).

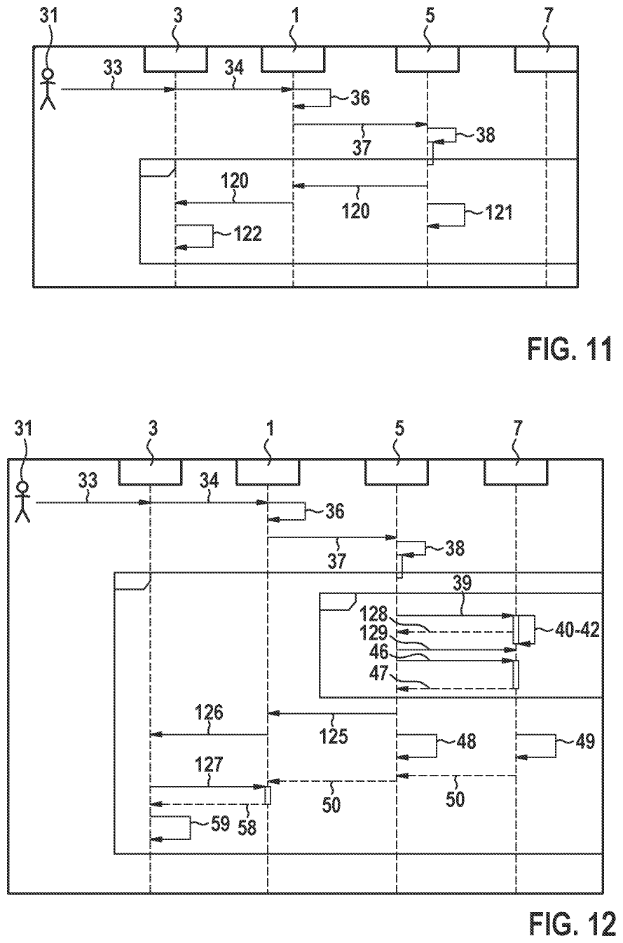

If the user rejects the update, which is shown in FIG. 11, the PR 3 will insert an RpPrStatus entity indicating the user rejected the update and a message is displayed to the PR 5 and CP 3 users via RMS 1 (steps 120). Accordingly, the UI of PR 3 and the CUI of CP 3 are updated (steps 121 and 122).

If the patient accepts the request in step 38 (see FIG. 10), the PR 3 sends the data to the medical device 7 using, for example, Bluetooth (step 39). The further steps 40 to 44 have already been explained with regards to FIG. 3. Upon completion the PR 5 will resynchronize with the medical device 7 (steps 46 and 47) and sends an RpPrStatus to the RMS 1 to indicate the result of the programming operation (step 125). If the follow-up ID or revision has changed, programming is considered to be successful, otherwise programming is determined to have failed. On successful programming, the CP 3 then receives an updated Follow-up entity from the RMS 1 which is loaded to continue the session (step 126). The following steps 58 and 59 have already been explained in connection with FIG. 3. If programming does not succeed, the CP 3 continues the session as it was prior to the “Transmit” button being pressed. The HCP uses the side menu end session option to end the session once all desired edits have been transmitted.

During remote programming, it is possible for the PR 3 to lose connection to the medical device 7 as indicated above which is depicted in FIG. 12 in step 128. In this case the resource manager module part 17e decides based on the current connectivity values that the communication module 17a of PR 3 shall first try to re-establish the communication connection using the same communication path. If the communication connection has been re-established (see step 129 in FIG. 12) and then resynchronization with the medical device 7 is provided. If after the completed resynchronization the follow-up ID or revision has changed, this will indicate successful programming. If the follow-up ID or revision has not changed the PR will retry the programming operation.

If, during remote programming, the CP 3 receives a RP 5 or medical device 7 update, or other entity, which indicates the follow-up ID or revision has changed to an unexpected value, the CP 3 will immediately notify the HCP and end the session. This scenario could occur if multiple CP's attempt to conduct remote programming sessions with the same PR 5 at the same time.

The system elements are specified (e.g., memory, processing capability, transmission speed, etc.) such that they are capable of performing within the boundary requirements of the connectivity use case of the particular embodiment. For example, in the RP embodiment, the elements must deliver at least the following system throughput requirements for input. These values are known by the respective resource manager module part 16e, 17e, 18e, 22e. If, for example, one actual communication connection cannot realize these requirements, the respective resource manager module part 16e, 17e, 18e, 22e may trigger switching to another communication path. The same applies to the hop-to-hop times and to the total times listed below.

Inputs

| | | | |
|---|---|---|---|
| LTE | Download | 22.69 | Mbps |
| (from PR's perspective) | | 2836.25 | KBps |
| PR's speeds based on Speedtest's United States “2017 Mobile | Upload | 8.51 | Mbps |
| Report” (based on Q1-Q2 2017 data) | | 1063.75 | KBps |
| http://www.speedtest.net/reports/united-states/#mobile | | | |
| BLE | Download | 344 | Kbps |
| (from medical device's perspective) | | 43 | KBps |
| The data rate is based on the assumption of 20 ms Connection | Upload | 344 | Kbps |
| Interval CI, the data payload of 215 bytes | | 43 | KBps |
| (MTU size of 251 bytes − 36 bytes of App Protocol | | | |
| Overhead), and 4 data packets per CI (in the case of | | | |
| Android). | | | |
| r = (I_MTU bytes * n_packets)/t_CI s | | | |
| RP Msg Processing Time | CP | 1 | s |
| | PR | 1 | s |
| | RMS | 15 | s |
| | External | 2 | s |
| | Push | | |
| | notification | | |
| | System | | |
| | TD* | 1 | s |

| | | |
|---|---|---|
| medical device BLE Message Processing Latency | 1 | s |
| RP CMD Payload Size | 3 | KB |
| Assuming this is the size of a single program (blob + implant data) | | |
| RP CMD Document Size | 4.5 | KB |
| (Payload + 50% Overhead) | | |
| medical device Interrogation Data Size | 36 | KB |
| Assuming PR interrogates all 12 programs after it | | |
| receives an RP Success msg. | | |
| RS RESP Document Size | 54 | KB |
| (payload + 50% Overhead) | | |
| *- Time it takes for TD to decrypt msg (in Mesquite), rule check program, | | |
| install program, activate program, and send alert to PR. | | |

Hop to Hop Time

| | | |
|---|---|---|
| RP CMD from CP to RMS | 1.00 | s |
| CP time to encrypt data + time to push data, for example, via Couchbase | | |
| (ignores Couchbase comm layer activity and takes simple approach of | | |
| datarate of data size). | | |
| RP CMD RMS Processing | 15.00 | s |
| Couchbase (for example) process duration + decrypting data using CP key + | | |
| encrypting data using medical device key + duration to send a push | | |
| notification via external service | | |
| RP CMD from RMS to PR | 3.00 | s |
| The time it takes for PR to receive push notification + time for PR to pull | | |
| data from RMS (ignores Couchbase comm layer activity and takes the | | |
| simple approach of transferring entire data packet based on transmission | | |
| rate) + PR processing | | |
| RP CMD from PR to medical device | 1.07 | s |
| BLE transmission time + medical device processing time (receive msg | | |
| decrypt msg, run rule check, install program) | | |
| PR Interrogate After RP Success Resp | 1.84 | s |
| Assuming PR reinterrogates all programs | | |
| RP RESP from PR to RMS | 1.05 | s |
| PR processing time + time to push data to RMS (assuming response sent | | |
| back to NSC contains latest therapeutic data returned from medical device; | | |
| ignores, e.g., Couchbase comm layer activity and takes the simple | | |
| approach of transferring entire data packet based on transmission rate) | | |
| RP RESP RMS Processing | 15.00 | s |
| Couchbase (e.g.) process duration + duration to send a push notification | | |
| via external service | | |
| RP RESP from RMS to CP | 3.02 | s |
| The time it takes for CP to receive push notification + time for CP to pull | | |
| data from RMS (ignores Couchbase comm layer activity and takes the | | |
| simple approach of transferring entire data packet based on transmission | | |
| rate) + CP processing | | |

Total Times

| | | |
|---|---|---|
| RP CMD from CP to PR | 19.01 | s |
| RP CMD PR to medical device + RP Install in TD | 1.07 | s |
| (after patient ack) | | |
| PR Interrogation of medical device + RP RESP PR to RMS + RP RESP | 20.91 | s |
| RMS to CP (after PR receives RP Success Resp) | | |
| Round Trip Time | 40.98 | s |
| (ignoring patient ack time) | | |

The system further allows combining subjective (e.g., patient diary, patient surveys, HCP notes) and objective data (e.g., current patient status, patient trends and outcomes analysis, longitudinal pain data, activity-based tracking, medication use checking, statistics) into one viewable RMS database via multiple data reporters, for example, CP 3, PR 5, RMS 1 (for example, NSC), Electronic Health Record system (EHR). Further, the RMS 1 provides a central service unit that serves as a central data repository of all of clinic's patients, as well as manages access rights and controls access to therapy device data according to user's login. The system may further comprise a web-based presentation layer, accessible from (1+N) different UI. The system may further provide HCP-accessible reporting which includes controlled access to database, configurable access rights within the database (i.e., to specific patient or groups of patients), accessible via multiple portals (CP, NSC UI, EHR UI, other). The HCP may view both current patient status as a real-time “snapshot” as well as via long-term data trends. The reporting may further comprise configurable report formats to allow customization to clinic or individual user's needs. The system further comprises a searchable patient and medical device database.

The RMS 1 may also have a user interface which may realize a closed loop, real-time communication with the at least one medical device 7. For example, if the HCP wants to know the patient's data in preparation for follow-up consult or remote assistance call with the patient the HCP triggers a respective request (update data request) from the RMS 1. This request is then transmitted to the PR 5 which in turn relays the request to the medical device 7. The medical device queries current patient and device status and transmits the report to the RMS 1 via the PR 5. The report is received and processed (i.e., the message is converted to form usable by the RMS 1, scanned for alert conditions using internal algorithms and assigned to the correct clinic and patient) by the RMS 1. Then, the HCP this able to review the new report in advance of the in-person meeting. The interface may be used to display data received via regular routine to connect data automatically (daily, monthly, . . . ) or on purpose (e.g., preparation for in-office visit, patient adjustment, . . . ). Further, the RMS 1 is configured to provide a current patient status ‘snapshot’ collected in near real-time showing all quantitative and subjective data store for the patient. In addition, as indicated above, the RMS 1 provides online access to the PR and the medical device to collect real time data. The RMS 1 is further configured to provide the status of the patient's system and statistics collected over time (e.g., for an SCS device implanted lead integrity, present stimulation settings, the diagnostic trends related to pain or system use). In one embodiment, the user may have the ability to configure the display of the combined status such that different data trends are visible or invisible according to the user's preferences. In order to improve efficiency of this connection, the system uses shorter transmission latencies and sends smaller data updates, more frequently, and/or with higher data resolution controlled by the resource manager module 16e, 17e, 18e, 22e, at the time HCP begins an interactive remote session with the patient. When the session ends the system reverts to larger data packet, less frequently, and/or with lower resolution.

In another embodiment, the HCP may be, in one embodiment, in the process of pulling up a patient record of RMS 1 using their CP 3 while enroute to see the patient in the Emergency Department. The session starts in the hospital lobby where good LTE connection is available, so the communication module 16a of CP 3 defaults to the LTE protocol based on the analysis of the present values of the connectivity parameters by the resource manager module part 16e. As they enter the Emergency Department, which has poor LTE but good WLAN with automatic connectivity enabled, the communication module 16a of CP 3 automatically switches the session to the WLAN network based on the changed connectivity parameters observed by the resource manager module 16e. In both cases it is important that the resource manager module 16e choses the above mentioned communication paths because these paths allow the HCP to stream the patient's data (e.g., an IEGM) in real time since the HCP's requirement is a real-time streaming where it is critical in an urgent situation to access patient's data (e.g., IEGM data) with near-zero latency.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A system having a network of connected elements comprising a remote monitoring server (RMS), at least one patient remote device (PR), at least one health care professional (HCP) remote device (CP), and at least one medical device,
wherein each of the elements of the system comprises a dedicated communication module, wherein the communication modules of the RMS, the at least one PR and the at least one CP are configured to establish and maintain a communication connection between each other, wherein the communication modules of the at least one medical device and of the at least one PR are configured to establish and maintain a communication connection between each other,
wherein each of the system's elements comprises a dedicated processing unit,
wherein each of the system's elements comprises a dedicated data memory module,
wherein each of the system's elements comprises a dedicated energy storage,
wherein the system provides resources used and shared by the communication modules, processing units, energy storages and/or data memory modules of the system's elements,
wherein the system further comprises a resource manager module configured to determine at least one resource of communication modules, processing units, data memory modules and/or energy storages involved in a user's and/or the system's at least one requirement, to analyze the determined resources and to automatically control the involved communication modules, involved processing units, involved energy storages and/or involved data memory modules based on the analysis of the determined resources, the current operating modes of the involved communication modules, involved processing units, involved energy storages and/or involved data memory modules and the user's and/or system's at least one requirement to be met.

2. The system of claim 1, wherein the resource of the communication modules of the system's elements comprise at least one connectivity parameter of at least one communication path through which the communication connections of the respective communication modules are provided, wherein connectivity parameters are, for example, network speed, ping time, packet loss percentage, transmission latency, transfer/transmission rate, signal strength, success rate of communication, resolution of transmitted data.

3. The system of claim 1, wherein the resource of the processing units of the system's elements comprises the load of the respective processing unit and/or at least one critical parameter.

4. The system of claim 1, wherein the resource of the data memory modules of the system's elements comprises the storage space of the respective data memory module.

5. The system of claim 1, wherein the resource of the energy storages of the system's elements comprises the charge level of the respective energy storage.

6. The system of claim 1, wherein the control of the resource manager module provides a feedback loop.

7. An operating method of a system having a network of connected elements comprising a remote monitoring server (RMS), at least one patient remote device (PR), at least one health care professional (HCP) remote device (CP), and at least one medical device,
wherein each of the elements of the system comprises a dedicated communication module, wherein the communication modules of the RMS, the at least one PR and the at least one CP establish and maintain a communication connection between each other, wherein the communication modules of the at least one medical device and of the at least one PR establish and maintain a communication connection between each other,
wherein each of the system's elements comprises a dedicated processing unit,
wherein each of the system's elements comprises a dedicated data memory module,
wherein each of the system's elements comprises a dedicated energy storage,
wherein resources are used and shared by the communication modules, processing units, energy storages and/or data memory modules of the system's elements,
wherein the system further comprises a resource manager module which determines at least one resource of communication modules, processing units, data memory modules and/or energy storages involved in a user's and/or the system's at least one requirement, analyzes the determined resources and automatically controls the involved communication modules, involved processing units, involved energy storages and/or involved data memory modules based on the analysis of the determined resources, the current operating modes of the involved communication modules, involved processing units, involved energy storages and/or involved data memory modules and the user's and/or system's at least one requirement to be met.

8. The method of claim 7, wherein the resource of the communication modules of the system's elements comprise at least one connectivity parameter of at least one communication path through which the communication connections of the respective communication modules are provided, wherein connectivity parameters are, for example, network speed, ping time, packet loss percentage, transmission latency, transfer/transmission rate, signal strength, success rate of communication, resolution of transmitted data.

9. The method of claim 7, wherein the resource of the processing units of the system's elements comprises the load of the respective processing unit and/or at least one critical parameter.

10. The method of claim 7, wherein the resource of the data memory modules of the system's elements comprises the storage space of the respective data memory module.

11. The method of claim 7, wherein the resource of the energy storages of the system's elements comprises the charge level of the respective energy storage.

12. The method of claim 7, wherein the control of the resource manager module provides a feedback loop.

13. A computer program product comprising instructions which, when executed by a processing unit, cause the processing unit to perform the steps of the method according to claim 7.

14. Computer readable data carrier storing a computer program product according to claim 13.
